(19) United States
(12) Patent Application Publication
(10) Pub. No.: US 2015/0344957 A1
(43) Pub. Date: Dec. 3, 2015

(54) MONITORING TREATMENT OF HISTIOCYTOSIS WITH VEMURAFENIB AND DABRAFENIB

(71) Applicant: TROVAGENE, INC., San Diego, CA (US)

(72) Inventors: Omar Abdel-Wahab, New York, NY (US); Elie L. Diamond, New York, NY (US); David M. Hyman, New York, NY (US); Filip Janku, Houston, TX (US); Mark G. Erlander, Carlsbad, CA (US); Cecile Rose T. Vibat, San Diego, CA (US)

(21) Appl. No.: 14/726,515

(22) Filed: May 30, 2015

Related U.S. Application Data
(60) Provisional application No. 62/006,261, filed on Jun. 1, 2014.
(60) Provisional application No. 62/040,368, filed on Aug. 21, 2014.

(51) Int. Cl. C12Q 1/68; A61K 31/506; A61K 31/437

(52) U.S. Cl. CPC C12Q 1/6883; A61K 31/437; A61K 31/506; C12Q 2600/156; C12Q 2600/106

(57) ABSTRACT

Provided are methods of determining the amount of a BRAF V600E mutation over time in a subject with Langerhans Cell Histiocytosis (LCH) or Erdheim-Chester Disease (ECD) who is being treated with vemurafenib or dabrafenib.

### CROSS-REFERENCE TO RELATED APPLICATIONS

This application claims the benefit of U.S. Provisional Application No. 62/006,261, filed Jun. 1, 2014, and U.S. Provisional Application No. 62/040,368, filed Aug. 21, 2014, both of which are incorporated by reference herein in its entirety.

### SEQUENCE LISTING

The instant application contains a Sequence Listing which has been submitted electronically in ASCII format and is hereby incorporated by reference in its entirety. Said ASCII copy, created on Jul. 7, 2015, is named 2-10319_SL.txt and is 10,887 bytes in size.

### BACKGROUND OF THE INVENTION

1. (1) Field of the Invention

The present application generally relates to methods of monitoring histiocytosis. More specifically, the invention provides assays for monitoring histiocytosis associated with the BRAF V600E mutation that is being treated with vemurafenib or dabrafenib.

2. (2) Description of the Related Art

Histiocytosis is a group of rare diseases characterized by the proliferation of histiocytes, or cells derived from monocytes, e.g., tissue macrophages and dendritic cells. Three groups of histiocytosis are recognized. One group is macrophage disorders, including hemophagocytic lymphohistiocytosis and Rosai-Dorfman Disease. The second group is malignant histiocytosis, and the third group is dendritic cell disorders, including Langerhans cell histiocytosis (LCH), juvenile xanthogranuloma, and Erdheim-Chester disease (ECD). ECD is a rare form of non-Langerhans cell histiocytosis affecting adults, which is associated with xanthogranulomatous infiltration of foamy macrophages (Janku et al., 2010, 2013; Arnaud et al., 2011),

The V600E mutation in BRAF is present in as many as 40-60% of patients with systemic histiocytosis, such as LCH and ECD (Badalian-Very et al., 2010; Haroche et al., 2013; Emile et al., 2013; Arceci, 2014).

A member of the serine/threonine kinase RAF family, the BRAF protein is part of the RAS-RAF-MEK-MAPK signaling pathway that plays a major role in regulating cell survival, proliferation and differentiation (Keshet and Seger, 2010). BRAF mutations constitutively activate the MEK-ERK pathway, leading to enhanced cell proliferation, survival and ultimately, neoplastic transformation (Wellbrock and Hurlstone, 2010; Niault and Baccarini, 2010).

Vemurafenib (ZELBORAF®), having the chemical formula N-{3-[5-(4-Chlorophenyl)-1H-pyrrolo[2,3-b]pyridin-3-carbonyl]-2,4-difluorophenyl}propane-1-sulfonamide, is a kinase inhibitor that can be effective in treating treat patients with histiocytosis where the BRAF V600E mutation is present (Haroche et al., 2012 and 2013). Another BRAF kinase inhibitor, Dabrafenib (TAFINLAR®) (N-{3-[5-(2-aminopyrimidin-4-yl)-2-tert-butyl-1,3-thiazol-4-yl]-2-fluorophenyl}-2,6-difluorobenzenesulfonamide) also holds promise as a treatment of histiocytosis associated with BRAF V600E.

After diagnosis by analysis of biopsy tissue and/or radiography, progression or remission of histiocytosis, particularly LCH, is generally followed radiographically or using other imaging techniques such as computed tomography (CT), MRI, or PET-CT. The present invention provides alternative or supplemental diagnostic and monitoring techniques for histiocytosis that are easier than imaging techniques and biopsy, are not invasive, do not subject the patient to radiation, and can provide additional data when the above clinical procedures are ambiguous.

### BRIEF SUMMARY OF THE INVENTION

The present invention is based in part on the discovery that treatment efficacy can be accurately determined in subjects with Langerhans Cell Histiocytosis (LCH) or Erdheim-Chester Disease (ECD) associated with a BRAF V600E mutation who are being treated with vemurafenib or dabrafenib by monitoring the BRAF V600E mutation in a bodily fluid of the subject.

Thus, in some embodiments, a method of determining the amount of a BRAF V600E mutation over time in a subject with LCH or ECD who is being treated with vemurafenib or dabrafenib is provided. The method comprises

(a) obtaining a first sample of a bodily fluid from the subject;

(b) amplifying, by PCR, a BRAF sequence encoding for BRAF amino acid 600 in a cell-free (cf) nucleic acid in the sample;

(c) determining the amount of BRAF V600E in the sample from the results of (b); and

(d) obtaining a second sample of a bodily fluid from the subject and repeating (b) and (c) at least once with the second sample of bodily fluid from the patient.

Also provided is a method of recommending a treatment for a subject with LCH or ECD associated with a BRAF V600E mutation who is being treated with vemurafenib or dabrafenib. The method comprises determining the amount of a BRAF V600E mutation in a first sample and a second sample of a bodily fluid from the subject by the above-described method; and

recommending continuing the treatment with vemurafenib or dabrafenib if the amount of BRAF V600E is similar to the amount of BRAF V600E in a similar sample from a patient with LCH or ECD and a BRAF V600E mutation which was effectively treated, or

recommending discontinuing the treatment with vemurafenib or dabrafenib if the amount of BRAF V600E is similar to the amount of BRAF V600E in a similar sample from a patient with LCH or ECD and a BRAF V600E mutation who has not been treated.

Further provided is a method of treating a subject with LCH or ECD associated with a BRAF V600E mutation who is being treated with vemurafenib or dabrafenib. The method comprises determining the amount of a BRAF V600E mutation in a first sample and a second sample of a bodily fluid from the subject by the above method; and

continuing the treatment with vemurafenib or dabrafenib if the amount of BRAF V600E is similar to the amount of BRAF V600E in a similar sample from a patient with LCH or ECD and a BRAF V600E mutation which was effectively treated, or

discontinuing the treatment with vemurafenib or dabrafenib if the amount of BRAF V600E is similar to the amount of BRAF V600E in a similar sample from a patient with LCH or ECD and a BRAF V600E mutation who has not been treated.

### BRIEF DESCRIPTION OF THE DRAWINGS

FIG. 1 illustrates the two-step assay design for a 28-30 bp footprint in the target gene sequence.

FIG. 2A and FIG. 2B show results from analysis of urinary and plasma cfDNA.

FIG. 3 shows decreased detection of mutant BRAF V600E with treated with vemurafenib or dabrafenib.

FIG. 4 shows results from monitoring six patients treated with vemurafenib.

FIG. 5 shows correlation between mutant BRAF V600E in urinary cfDNA and radiographic response to treatment with dabrafenib.

FIG. 6 shows correlation between mutant BRAF V600E in urinary cfDNA and radiographic response to treatment with vemurafenib.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

The present invention is based in part on the discovery, e.g., as described in the Examples and figures, that treatment efficacy can be accurately determined in subjects with Langerhans Cell Histiocytosis (LCH) or Erdheim-Chester Disease (ECD) associated with a BRAF V600E mutation, who are being treated with vemurafenib or dabrafenib, by monitoring the BRAF V600E mutation in a bodily fluid of the subject. That discovery allows for the determination of treatment efficacy as an adjunct to, or substitute for, the more invasive and dangerous methods used to make those determinations, e.g., radiography and biopsy.

Thus, in some embodiments, a method of determining the amount of a BRAF V600E mutation over time in a subject with histiocytosis who is being treated with vemurafenib or dabrafenib is provided. The method comprises

(a) obtaining a first sample of a bodily fluid from the subject;

(b) amplifying, by PCR, a BRAF sequence encoding for BRAF amino acid 600 in a cell-free (cf) nucleic acid in the sample;

(c) determining the amount of BRAF V600E in the sample from the results of (b); and

(d) obtaining a second sample of a bodily fluid from the subject and repeating (b) and (c) at least once with the second sample of bodily fluid from the patient.

While these methods would be expected to be effective in a subject having any activating mutation in BRAF, the V600E mutation is by far the most predominant. That mutation, in which a glutamic acid (Glu or E) is substituted for a Valine (Val or V) residue at position or amino acid residue 600 of SEQ ID NO: 2. Alternatively, or in addition, the BRAF mutation is a substitution of an adenine (A) for a thymine (T) nucleotide at position 1860 of SEQ ID NO: 1, which is the most common mutation that causes the V600E mutant BRAF protein.

Wildtype Homo sapiens v-raf murine sarcoma viral oncogene homolog B1, BRAF, is encoded by the following mRNA sequence (NM_{—}004333, SEQ ID NO:1) (wherein coding sequence is bolded and the coding sequence for amino acid residue 600 is underlined and enlarged):

| | |
|---|---|
| 1 | cgcctccctt ccccctcccc gcccgacagc ggccgctcgg gccccggctc tcggttataa |
| 61 | gatggcggcg ctgagcggtg gcggtggtgg cggcgcggag ccgggccagg ctctgttcaa |
| 121 | cggggacatg gagcccgagg ccggcgccgg cgccggcgcc gcggcctctt cggctgcgga |
| 181 | ccctgccatt ccggaggagg tgtggaatat caaacaaatg attaagttga cacaggaaca |
| 241 | tatagaggcc ctattggaca aatttggtgg ggagcataat ccaccatcaa tatatctgga |
| 301 | ggcctatgaa gaatacacca gcaagctaga tgcactccaa caaagagaac aacagttatt |
| 361 | ggaatctctg gggaacggaa ctgatttttc tgtttctagc tctgcatcaa tggataccgt |
| 421 | tacatcttct tcctcttcta gcctttcagt gctaccttca tctctttcag tttttcaaaa |
| 481 | tcccacagat gtggcacgga gcaaccccaa gtcaccacaa aaacctatcg ttagagtctt |
| 541 | cctgcccaac aaacagagga cagtggtacc tgcaaggtgt ggagttacag tccgagacag |
| 601 | tctaaagaaa gcactgatga tgagaggtct aatcccagag tgctgtgctg tttacagaat |
| 661 | tcaggatgga gagaagaaac caattggttg ggacactgat atttcctggc ttactggaga |
| 721 | agaattgcat gtggaagtgt tggagaatgt tccacttaca acacacaact ttgtacgaaa |
| 781 | aacgtttttc accttagcat tttgtgactt ttgtcgaaag ctgcttttcc agggtttccg |
| 841 | ctgtcaaaca tgtggttata aatttcacca gcgttgtagt acagaagttc cactgatgtg |
| 901 | tgttaattat gaccaacttg atttgctgtt tgtctccaag ttctttgaac accacccaat |
| 961 | accacaggaa gaggcgtcct tagcagagac tgccctaaca tctggatcat ccccttccgc |
| 1021 | acccgcctcg gactctattg ggccccaaat tctcaccagt ccgtctcctt caaaatccat |
| 1081 | tccaattcca cagcccttcc gaccagcaga tgaagatcat cgaaatcaat ttgggcaacg |
| 1141 | agaccgatcc tcatcagctc ccaatgtgca tataaacaca atagaacctg tcaatattga |
| 1201 | tgacttgatt agagaccaag gatttcgtgg tgatggagga tcaaccacag gtttgtctgc |
| 1261 | taccccccct gcctcattac ctggctcact aactaacgtg aaagccttac agaaatctcc |
| 1321 | aggacctcag cgagaaagga agtcatcttc atcctcagaa gacaggaatc gaatgaaaac |
| 1381 | acttggtaga cgggactcga gtgatgattg ggagattcct gatgggcaga ttacagtggg |
| 1441 | acaaagaatt ggatctggat catttggaac agtctacaag ggaaagtggc atggtgatgt |
| 1501 | ggcagtgaaa atgttgaatg tgacagcacc tacacctcag cagttacaag ccttcaaaaa |
| 1561 | tgaagtagga gtactcagga aaacacgaca tgtgaatatc ctactcttca tgggctattc |
| 1621 | cacaaagcca caactggcta ttgttaccca gtggtgtgag ggctccagct tgtatcacca |
| 1681 | tctccatatc attgagacca aatttgagat gatcaaactt atagatattg cacgacagac |
| 1741 | tgcacagggc atggattact tacacgccaa gtcaatcatc cacagagacc tcaagagtaa |
| 1801 | taatatattt cttcatgaag acctcacagt aaaaataggt gattttggtc tagctacagt |
| 1861 | gaaatctcga tggagtgggt cccatcagtt tgaacagttg tctggatcca ttttgtggat |
| 1921 | ggcaccagaa gtcatcagaa tgcaagataa aaatccatac agctttcagt cagatgtata |
| 1981 | tgcatttgga attgttctgt atgaattgat gactggacag ttaccttatt caaacatcaa |
| 2041 | caacagggac cagataattt ttatggtggg acgaggatac ctgtctccag atctcagtaa |
| 2101 | ggtacggagt aactgtccaa aagccatgaa gagattaatg gcagagtgcc tcaaaaagaa |
| 2161 | aagagatgag agaccactct ttccccaaat tctcgcctct attgagctgc tggcccgctc |
| 2221 | attgccaaaa attcaccgca gtgcatcaga accctccttg aatcgggctg gtttccaaac |
| 2281 | agaggatttt agtctatatg cttgtgcttc tccaaaaaca cccatccagg cagggggata |
| 2341 | tggtgcgttt cctgtccact gaaacaaatg agtgagagag ttcaggagag tagcaacaaa |
| 2401 | aggaaaataa atgaacatat gtttgcttat atgttaaatt gaataaaata ctctcttttt |
| 2461 | ttttaaggtg aaccaaagaa cacttgtgtg gttaaagact agatataatt tttccccaaa |
| 2521 | ctaaaattta tacttaacat tggattttta acatccaagg gttaaaatac atagacattg |
| 2581 | ctaaaaattg gcagagcctc ttctagaggc tttactttct gttccgggtt tgtatcattc |
| 2641 | acttggttat tttaagtagt aaacttcagt ttctcatgca acttttgttg ccagctatca |
| 2701 | catgtccact agggactcca gaagaagacc ctacctatgc ctgtgtttgc aggtgagaag |
| 2761 | ttggcagtcg gttagcctgg gttagataag gcaaactgaa cagatctaat ttaggaagtc |
| 2821 | agtagaattt aataattcta ttattattct taataatttt tctataacta tttcttttta |
| 2881 | taacaatttg gaaaatgtgg atgtctttta tttccttgaa gcaataaact aagtttcttt |
| 2941 | taaaaa |

Wildtype Homo sapiens v-raf murine sarcoma viral oncogene homolog B1, BRAF, is encoded by the following amino acid sequence (NP_{—}004324, SEQ ID NO: 2) (wherein amino acid residue 600 is bolded and underlined and enlarged):

| | |
|---|---|
| 1 | maalsggggg gaepgqalfn gdmepeagag agaaassaad paipeevwni kqmikltqeh |
| 61 | iealldkfgg ehnppsiyle ayeeytskld alqqreqqll eslgngtdfs vsssasmdtv |
| 121 | tsssssslsv lpsslsvfqn ptdvarsnpk spqkpivrvf lpnkqrtvvp arcgvtvrds |
| 181 | lkkalmmrgl ipeccavyri qdgekkpigw dtdiswltge elhvevlenv pltthnfvrk |
| 241 | tfftlafcdf crkllfqgfr cqtcgykfhq rcstevplmc vnydqldllf vskffehhpi |
| 301 | pqeeaslaet altsgsspsa pasdsigpqi ltspspsksi pipqpfrpad edhrnqfgqr |
| 361 | drsssapnvh intiepvnid dlirdqgfrg dggsttglsa tppaslpgsl tnvkalqksp |
| 421 | gpqrerksss ssedrnrmkt lgrrdssddw eipdgqitvg qrigsgsfgt vykgkwhgdv |
| 481 | avkmlnvtap tpqqlqafkn evgvlrktrh vnillfmgys tkpqlaivtq wcegsslyhh |
| 541 | lhiietkfem iklidiarqt aqgmdylhak siihrdlksn niflhedltv kigdfglatv |
| 601 | ksrwsgshqf eqlsgsilwm apevirmqdk npysfqsdvy afgivlyelm tgqlpysnin |
| 661 | nrdqiifmvg rgylspdlsk vrsncpkamk rlmaeclkkk rderplfpqi lasiellars |
| 721 | lpkihrsase pslnragfqt edfslyacas pktpiqaggy gafpvh |

In various embodiments of the methods described herein, the subjects are humans. The subjects may be of any age, including, but not limited to infants, toddlers, children, minors, adults, seniors, and elderly individuals. In some embodiments, the histiocytosis is Langerhans Cell Histiocytosis (LCH). In other embodiments, the histiocytosis is a non-Langerhans Cell Histiocytosis (nLCH). In some of these embodiments, the nLCH is Erdheim-Chester Disease (ECD). Other non-limiting examples of an nLCH include benign cephalic histiocytosis, generalized eruptive histiocytoma, (giant cell) reticulohistiocytoma, hemophagocytic lymphohistiocytosis (HLH), indeterminate cell histiocytosis, juvenile xanthogranuloma (JXG), Kikuchi disease, multicentric reticulohistiocytosis, necrobiotic xanthogranuloma, Niemann-Pick disease, progressive nodular histiocytoma, Rosai-Dorfman disease, Sea-blue histiocytosis, and xanthoma disseminatum. Other possible examples are interdigitating dendritic sarcoma and histiocytic sarcoma. Thus, the histiocytosis can be cancerous or noncancerous.

The term “LCH” or Langerhans Cell Histiocytosis is intended to encompass the same condition that may be identified by other names, such as Abt-Letterer-Si we disease, Eosinophilic Granuloma, Hand-Schuller-Christian Disease, Letterer-Siwe Disease, and Histiocytosis X.

In any of the methods described herein, the amount of the mutation can be determined by any method known in the art. Nonlimiting examples include MALDI-TOF, HR-melting, di-deoxy-sequencing, single-molecule sequencing, use of probes, pyrosequencing, second generation high-throughput sequencing, SSCP, RFLP, dHPLC, CCM, or methods utilizing the polymerase chain reaction (PCR), e.g., digital PCR, quantitative-PCR, or allele-specific PCR (where the primer or probe is complementary to the variable gene sequence). In some embodiments, the PCR is droplet digital PCR, e.g., as described in the Examples. In some of these methods, the mutation is quantified along with the wildtype sequence, to determine the percentage of mutated sequence. In other methods, only the mutation is quantified. In the latter case,

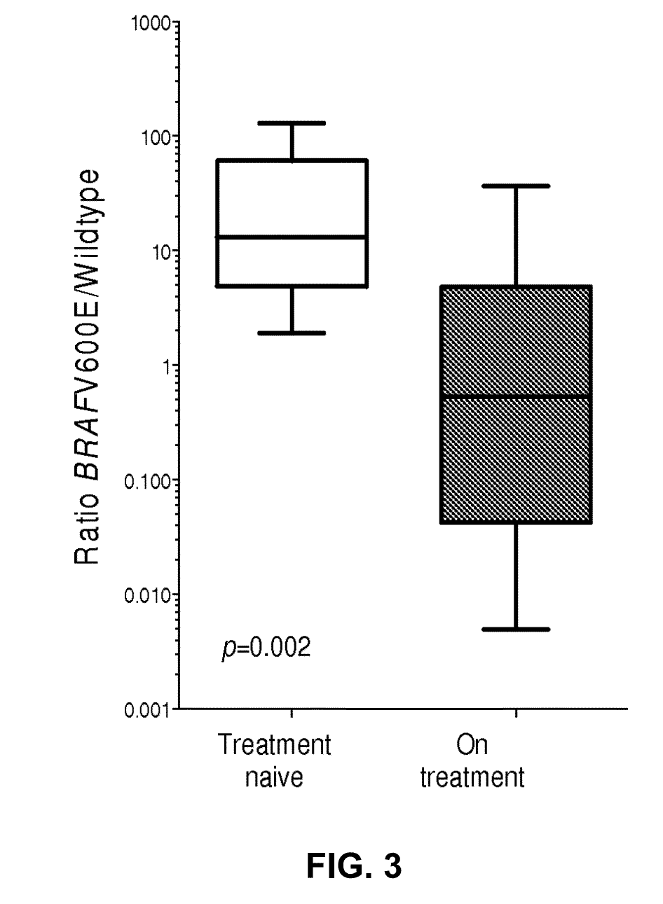

In embodiments where both the mutation and the wildtype BRAF sequence is determined, the ratio of BRAF V600E to wild-type BRAF may be calculated, where effective treatment with vemurafenib or dabrafenib would be expected to result in a lower ratio and ineffective treatment would be expected to result in a higher ratio. See, e.g., Examples and FIGS. 3A, 3B, and 4-7. As described therein, a blocking oligonucleotide may be included in the amplifying step (b) to reduce the amount of wild-type BRAF that is amplified.

As shown in the Examples, the ratio of BRAF V600E to wild-type BRAF is calculated, can range from about 0.01 to more than 100, where effectively treated subjects would have a ratio of 0.01 to about 5, and subjects that were not effectively treated, or before treatment can range from about 3 to more than 100. I would be appreciated that the ratio from effectively treated vs. ineffectively treated can be different among assays. Thus, for any particular assay, the cutoff between effectively and ineffectively treated can vary, and that cutoff should be determined before and/or during the assay using standard samples of known effectiveness, as is known in the art.

The detection limits (i.e., cutoff) for the presence of a BRAF mutation in cf nucleic acids may be determined by assessing data from one or more of negative controls (e.g. from healthy control subjects or verified cell lines) and a plurality of patient samples. Optionally, the limits may be determined based in part on minimizing the percentage of false negatives as being more important than minimizing false positives, or vice versa. One set of non-limiting thresholds for BRAF V600E is defined as less than about 0.05% of the mutation in a sample of cf nucleic acids for a determination of no mutant present or wild-type only; the range of about 0.05% to about 0.107% as “borderline”, and greater than about 0.107% as detected mutation. In other embodiments, and with other mutations, a no-detection designation threshold for the mutation is set at less than about 0.2%, less than about 0.3%, less than about 0.4%, less than about 0.5%, less than about 0.6%, less than about 0.7%, less than about 0.8%, less than about 0.9%, less than about 1%, less than about 2%, less than about 3%, less than about 4%, or less than about 5% detection of the mutation relative to a corresponding wildtype sequence. Of course the inclusion of additional patient samples may result in the determination of different threshold values for each category, or alternatively the inclusion or elimination of the “borderline” category. The desired amount of false negatives to false positives will also have an effect on the threshold value.

In many embodiments, the nucleic acids are cf DNA (“cfDNA”). In some embodiments, the amplified or detected DNA molecule is genomic DNA. In other embodiments, the amplified or detected molecule is a cDNA. In other embodiments, the nucleic acids is cfRNA or cf mRNA.

Any bodily fluid that would be expected to have cfDNA can be utilized in these methods. Non-limiting examples of a bodily fluid include, but are not limited to, peripheral blood, serum, plasma, urine, lymph fluid, amniotic fluid, and cerebrospinal fluid. In certain particular embodiments, such as those illustrated in the Examples, the bodily fluid is blood, serum, plasma or urine.

The disclosed methods may be repeated two or more times to provide measurements over time. In some cases, the methods are repeated three or more times, four or more times, five or more times, or six or more times. The repetition of the methods may be at regular intervals, or at irregular intervals. Non-limiting examples of intervals include biweekly and monthly. In some embodiments, the first sample is obtained after diagnosis of LCH or ECD but before or at commencement of treatment with vemurafenib or dabrafenib.

In some aspects, the monitoring of the mutation is accompanied by a determining the disease burden, e.g., by radiography, computed tomography (CT) scanning, positron emission tomography (PET), or PET/CT scanning, and comparing the determined amount of mutation to the disease burden. This can be useful to determine whether, or confirm that the mutation being monitored is actually the driver of the disease.

In other aspects, the determined amount of mutation is not compared to disease burden, either at one, more than one, or all the mutation monitoring times. Given the reliability of the mutation monitoring procedures described herein, a disease burden assessment need not be made at each time point, thus saving the patient a disease burden assessment.

Thus, these methods may be used to confirm the maintenance of a disclosed treatment or therapy against histiocytosis, or to change the treatment or therapy against the disease. Within the scope of changing treatment or therapy, the disclosure includes increasing the treatment or therapy; reducing the treatment or therapy, optionally to the point of terminating the treatment or therapy; terminating the treatment or therapy with the start of another treatment or therapy; and adjusting the treatment or therapy as non-limiting examples. Non-limiting examples of adjusting the treatment or therapy include reducing or increasing the therapy, optionally in combination with one or more additional treatments or therapies; or maintaining the treatment or therapy while adding one or more additional treatments or therapies.

In some cases, the observation of cell-free (cf) nucleic acids identifies an increase in the levels of cf nucleic acids containing the mutation following the start of a treatment or therapy. Following the increase, the observation may reach an inflection point, where the levels decrease, or continue to increase. The presence of an inflection point may be used to determine responsiveness to the treatment or therapy, which may be maintained or reduced. A continuing decrease in the levels to be the same as, or lower than, the levels before the start of treatment of therapy is a further confirmation of responsiveness.

The absence of an inflection point indicates resistance to the treatment or therapy and so may be followed by terminating administration of the treatment or therapy, or administering at least one additional treatment or therapy against the disease or disorder to the patient, reducing the treatment of the subject with the treatment or therapy and administering at least one additional treatment or therapy against the disease or disorder to the subject.

In other cases, and following an inflection point and a decrease in levels, an additional inflection point may be observed. This may indicate the development of resistance to the treatment or therapy and be followed by terminating administration of the treatment or therapy, or administering at least one additional treatment or therapy against the disease or disorder to the subject, or reducing the treatment of the subject with the therapy and administering at least one additional therapy against the disease or disorder to the subject.

Also provided is a method of recommending a treatment for a subject with LCH or ECD associated with a BRAF V600E mutation who is being treated with vemurafenib or dabrafenib. The method comprises determining the amount of a BRAF V600E mutation in a first sample and a second sample of a bodily fluid from the subject by the above-described method; and

recommending continuing the treatment with vemurafenib or dabrafenib if the amount of BRAF V600E is similar to the amount of BRAF V600E in a similar sample from a patient with LCH or ECD and a BRAF V600E mutation which was effectively treated, or

recommending discontinuing the treatment with vemurafenib or dabrafenib if the amount of BRAF V600E is similar to the amount of BRAF V600E in a similar sample from a patient with LCH or ECD and a BRAF V600E mutation who has not been treated.

In some embodiments, the first sample is obtained after diagnosis of LCH or ECD but before or at commencement of treatment with vemurafenib or dabrafenib and

recommending continuing the treatment with vemurafenib or dabrafenib if the amount of BRAF V600E in the second sample is significantly lower than the amount of BRAF V600E in the first sample, or

recommending discontinuing the treatment with vemurafenib or dabrafenib if the amount of BRAF V600E in the second sample is not significantly lower than the amount of BRAF V600E in the first sample.

Further provided is a method of treating a subject with LCH or ECD associated with a BRAF V600E mutation who is being treated with vemurafenib or dabrafenib. The method comprises determining the amount of a BRAF V600E mutation in a first sample and a second sample of a bodily fluid from the subject by the above method; and

continuing the treatment with vemurafenib or dabrafenib if the amount of BRAF V600E is similar to the amount of BRAF V600E in a similar sample from a patient with LCH or ECD and a BRAF V600E mutation which was effectively treated, or

discontinuing the treatment with vemurafenib or dabrafenib if the amount of BRAF V600E is similar to the amount of BRAF V600E in a similar sample from a patient with LCH or ECD and a BRAF V600E mutation who has not been treated.

In some embodiments, the first sample is obtained after diagnosis of LCH or ECD before or at commencement of treatment with vemurafenib or dabrafenib and

continuing the treatment with vemurafenib or dabrafenib if the amount of BRAF V600E in the second sample is significantly lower than the amount of BRAF V600E in the first sample, or

discontinuing the treatment with vemurafenib or dabrafenib if the amount of BRAF V600E in the second sample is not significantly lower than the amount of BRAF V600E in the first sample.

As used herein, a “subject” includes a mammal. The mammal can be e.g., any mammal, e.g., a human, primate, bird, mouse, rat, fowl, dog, cat, cow, horse, goat, camel, sheep or a pig. In many cases, the mammal is a human being.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), and Remington's Pharmaceutical Sciences, Mack Publishing Co., Easton, Pa., 18th edition (1990). These texts can, of course, also be referred to in making or using an aspect of the disclosure.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### Materials and Methods

The following methods described herein were utilized in the examples that follow.

### Patient Urine Samples

Between January 2013 and June 2014, 26 consecutive patients with LCH (n=5) and ECD (n=21) were enrolled from Memorial Sloan Kettering Cancer Center and MD Anderson Cancer Center. Urinary cell-free (cf)DNA analysis was performed in all patients and plasma cfDNA analysis in 19/26 patients. Serial urinary samples in 10 BRAF V600E mutant samples were also obtained to track disease burden with therapy. Urine and plasma cfDNA were quantified by a droplet digital PCR (ddPCR; QX-100, BioRad).

### Two-Step Assay Design

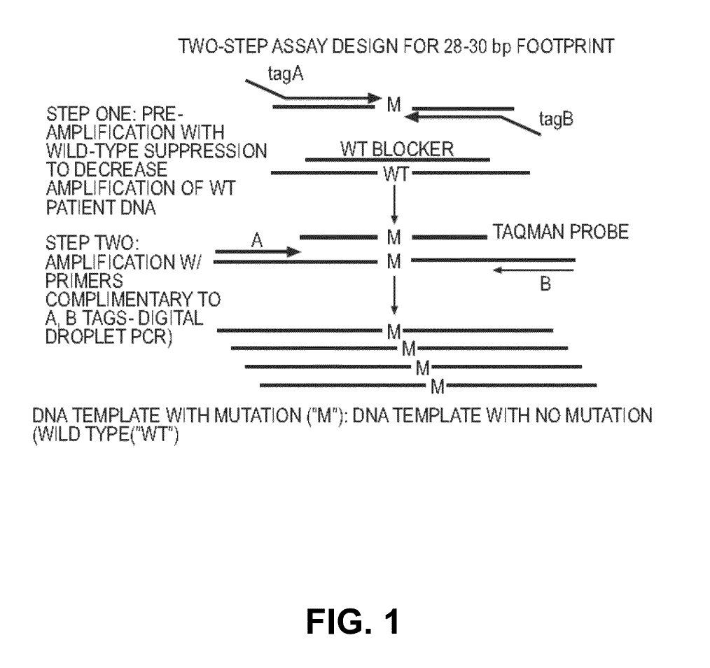

A two-step assay design was developed for a 28-30 basepair footprint in the target mutant gene sequence. FIG. 1 summarizes the assay design, which includes a first preamplification step to increase the number of copies of a target mutant gene sequence relative to wild-type gene sequences that are present in the sample. The pre-amplification is conducted in the presence of a wild-type (non-mutant) suppressing “WT blocker” oligonucleotide that is complementary to the wild-type sequence (but not the mutant sequence) to decrease amplification of wild-type DNA. The pre-amplification is performed with primers that include adapters (or “tags”) at the 5′ end to facilitate amplification in the second step.

The second step is additional amplification with primers complementary to the tags on the ends of the primers used in the first step and a TaqMan (reporter) probe oligonucleotide complementary to the mutant sequence for quantitative, digital droplet PCR.

### Concordance

Of 26 patients, initial tissue BRAF V600E genotyping identified 11 patients to be BRAF V600E mutant (42.3%); 6 patients as BRAF wildtype (23.1%); 9 patients with indeterminate initial tissue biopsy result (34.6%). Concordance between urinary cfDNA, plasma cfDNA, and tissue biopsy BRAF V600E genotyping results is shown in Tables 1 and 2.

TABLE 1
Concordance between urinary cfDNA and tissue biopsy for presence of
BRAF V600E in histiocytosis patients.

| | Tissue | Tissue | Tissue | Urine |
|---|---|---|---|---|
| | biopsy | biopsy | biopsy | cfDNA |
| | (+) | (−) | (?) | total |
| Urine cfDNA (+) | 10 | 0 | 4 | 14 |
| Urine cfDNA (−) | 1* | 6 | 4 | 11 |
| Urine cfDNA (?) | 0 | 0 | 1 | 1 |
| Tissue biopsy total | 11 | 6 | 9 | |

*Urine sample for this patient was acquired during therapy while tissue biopsy was performed pre-treatment
? Indeterminate

TABLE 2
Concordance between plasma cfDNA and tissue biopsy for presence of
BRAF V600E in histiocytosis patients.

| | Tissue | Tissue | Tissue | Urine |
|---|---|---|---|---|
| | biopsy | biopsy | biopsy | cfDNA |
| | (+) | (−) | (?) | total |
| Plasma cfDNA (+) | 8 | 1 | 2* | 10 |
| Plasma cfDNA (−) | 0 | 3 | 5 | 9 |
| Plasma cfDNA (?) | 0 | 0 | 0 | 0 |
| Tissue biopsy total | 8 | 4 | 7 | |

*These 2 patients subsequently underwent repeat tissue biopsy which confirmed cfDNA test result and allowed the patients to enroll in phase II study of vemurafenib
? Indeterminate

### Example 2

### BRAF V600E Mutations in cfDNA

Quantitative analyses of the BRAF V600E mutation in urinary and plasma cfDNA reliably detects BRAF V600E mutation based on tissue genotype. The analyses in FIGS. 3A (urinary cfDNA) and 3B (plasma cfDNA) include plasma and urine samples pretreatment and on treatment.

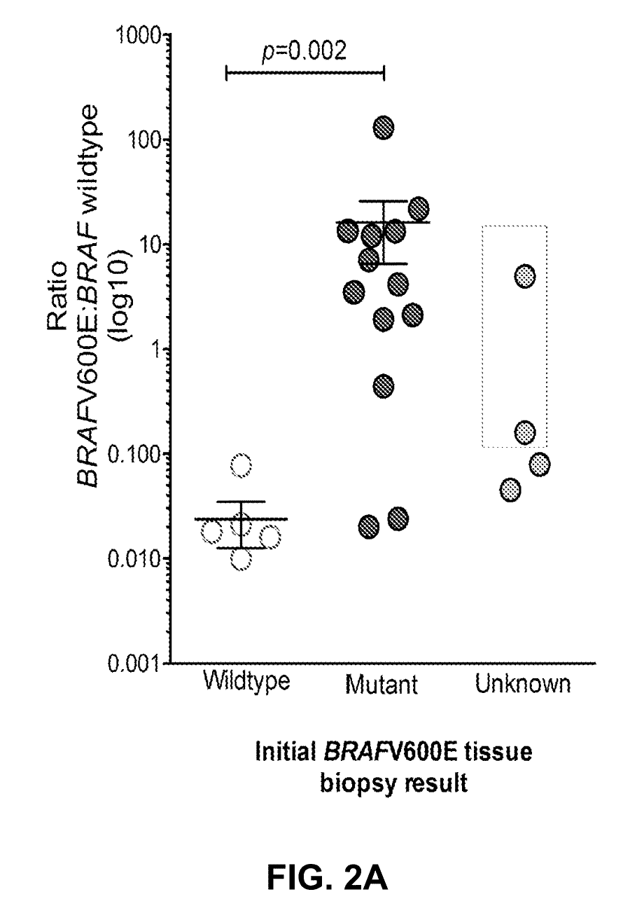

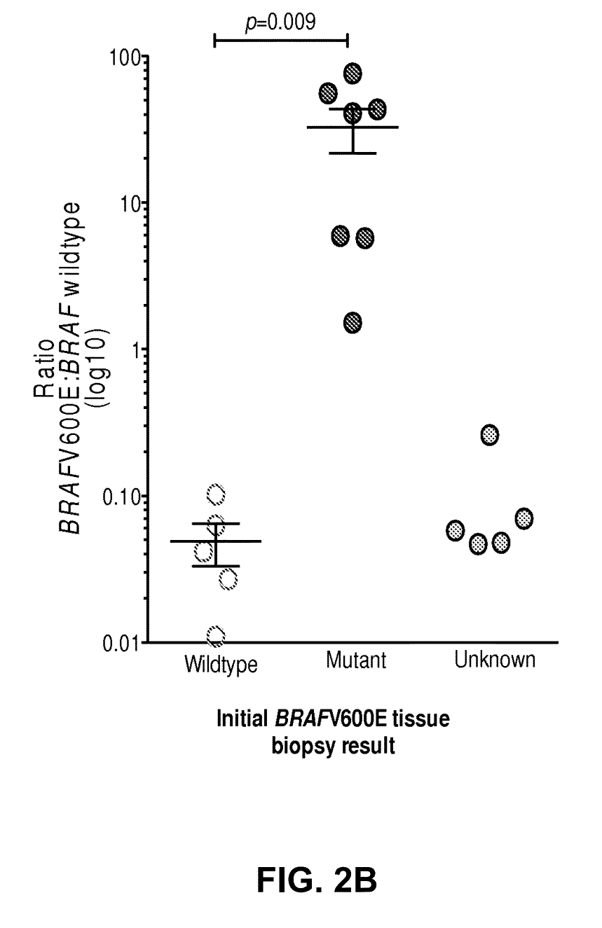

Two patients with an indeterminate biopsy result but with clearly positive urinary cfDNA BRAF V600E mutation subsequently were found to have BRAF V600E mutation in tissue with a repeat biopsy (box in FIG. 2A).

FIG. 3 shows that quantitative cfDNA analysis of BRAF V600E burden in urine in treatment naïve versus treated patient samples detects decreased mutational burden on therapy.

### Example 3

### Monitoring of Treatment with Vemurafenib

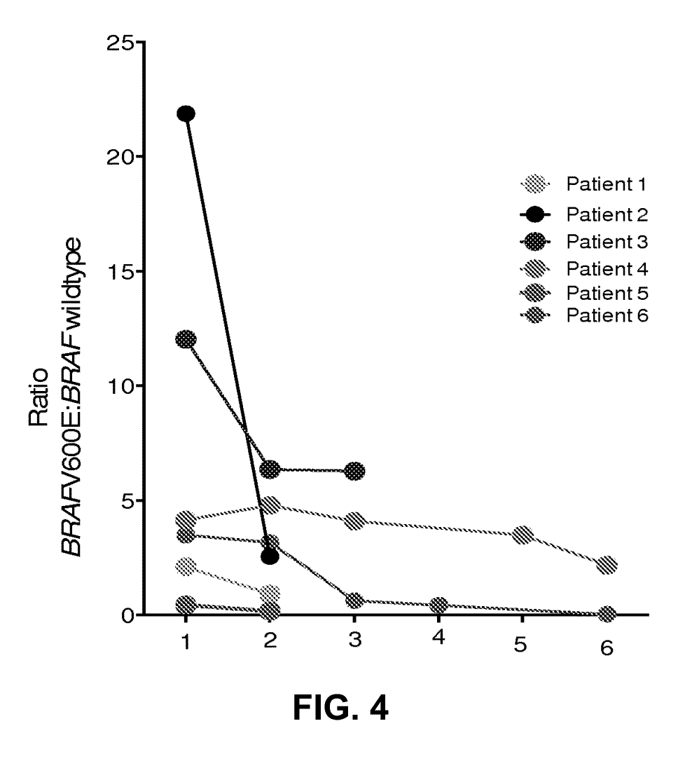

Analysis over time of BRAF V600E mutation in urinary cfDNA of patients treated with vemurafenib reveals decreased BRAF mutant allele burden with therapy. See FIG. 4 for data from initial measurement to up to five subsequent time points.

### Example 4

### Correlation with Radiographic Response

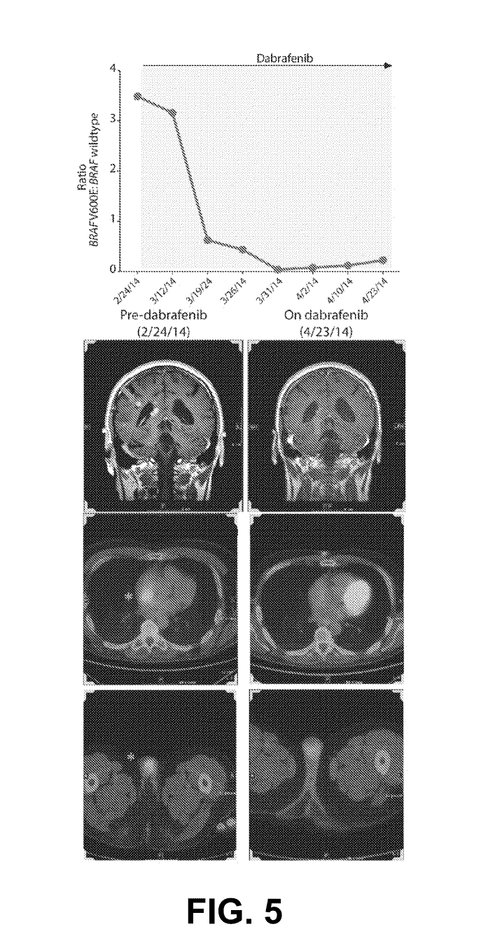

FIG. 5 shows that BRAF V600E mutation burden in urine of subjects treated with dabrafenib correlates with radiographic response. From an initial untreated state, urine samples from a subject treated with dabrafenib displayed a decrease in the mutation in cfDNA during the course of treatment.

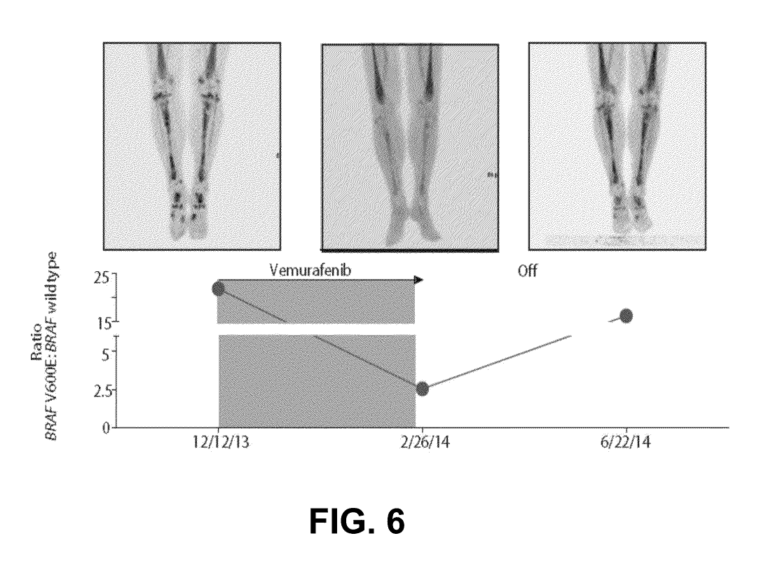

FIG. 6 shows that BRAF V600E mutation burden in urine of subjects treated with vemurafenib correlates with radiographic response. A subject was treated with vemurafenib, which resulted in a decrease in the mutation in urinary cfDNA from the subject. The end of treatment showed an increase, or “rebound,” in the amount of the mutation in urinary cfDNA. Therefore, the BRAF V600E mutation burden in urine of subjects is observed to change dynamically with therapy.

### REFERENCES

Arceci R J, 2014, Am Soc Clin Oncol Educ Book. 2014:e441-5.
Arnaud et al., 2011, Blood 117:2778-2782.
Badalian-Very et al., 2010, Blood 116:1919-23.
Emile et al., 2013, J Clin Oncol. 31:398.
Haroche et al., 2012, Blood 120:2700-3.
Haroche et al., 2013, Blood 121:1495-1500.
Janku et al., 2010, J Clin Oncol. 28:e633-636.
Janku et al., 2013, Oncologist 18:2-4.
Keshet Y and Seger R., 2010, Methods Mol Biol. 661:3-38.
Niault T and Baccarini M., 2010, Carcinogenesis. 31:1165-74.
Wellbrock C and Hurlstone A, 2010, Pharmacol. 80:561-7.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of determining the amount of a BRAF V600E mutation over time in a subject with Langerhans Cell Histiocytosis (LCH) or Erdheim-Chester Disease (ECD) who is being treated with vemurafenib or dabrafenib, the method comprising
(a) obtaining a first sample of a bodily fluid from the subject;
(b) amplifying, by PCR, a BRAF sequence encoding for BRAF amino acid 600 in a cell-free (cf) nucleic acid in the sample;
(c) determining the amount of BRAF V600E in the sample from the results of (b); and
(d) obtaining a second sample of a bodily fluid from the subject and repeating (b) and (c) at least once with the second sample of bodily fluid from the patient.

2. The method of claim 1, wherein the bodily fluid is urine, blood, serum, or plasma.

3. The method of claim 1, wherein the cf nucleic acid is DNA.

4. The method of claim 1, wherein both BRAF V600E and wild-type BRAF is amplified and the ratio of BRAF V600E to wild-type BRAF is determined.

5. The method of claim 1, wherein a blocking oligonucleotide is included in the amplifying step (b) to reduce the amount of wild-type BRAF that is amplified.

6. The method of claim 1, wherein the subject has ECD.

7. The method of claim 1, wherein the subject has LCH.

8. The method of claim 1, wherein the amount of BRAF V600E is determined with quantitative PCR or sequencing.

9. The method of claim 8, wherein the amount of BRAF V600E is determined with droplet digital PCR.

10. The method of claim 9, wherein the repeating is for two or more times.

11. The method of claim 1, wherein the first sample is obtained after diagnosis of LCH or ECD before or at commencement of treatment with vemurafenib or dabrafenib.

12. A method of recommending a treatment for a subject with LCH or ECD associated with a BRAF V600E mutation who is being treated with vemurafenib or dabrafenib, the method comprising determining the amount of a BRAF V600E mutation in a first sample and a second sample of a bodily fluid from the subject by the method of claim 1; and
recommending continuing the treatment with vemurafenib or dabrafenib if the amount of BRAF V600E is similar to the amount of BRAF V600E in a similar sample from a patient with LCH or ECD and a BRAF V600E mutation which was effectively treated, or
recommending discontinuing the treatment with vemurafenib or dabrafenib if the amount of BRAF V600E is similar to the amount of BRAF V600E in a similar sample from a patient with LCH or ECD and a BRAF V600E mutation who has not been treated.

13. The method of claim 12, wherein the first sample is obtained after diagnosis of LCH or ECD but before or at commencement of treatment with vemurafenib or dabrafenib and
recommending continuing the treatment with vemurafenib or dabrafenib if the amount of BRAF V600E in the second sample is significantly lower than the amount of BRAF V600E in the first sample, or
recommending discontinuing the treatment with vemurafenib or dabrafenib if the amount of BRAF V600E in the second sample is not significantly lower than the amount of BRAF V600E in the first sample.

14. A method of treating a subject with LCH or ECD associated with a BRAF V600E mutation who is being treated with vemurafenib or dabrafenib, the method comprising determining the amount of a BRAF V600E mutation in a first sample and a second sample of a bodily fluid from the subject by the method of claim 1; and
continuing the treatment with vemurafenib or dabrafenib if the amount of BRAF V600E is similar to the amount of BRAF V600E in a similar sample from a patient with LCH or ECD and a BRAF V600E mutation which was effectively treated, or
discontinuing the treatment with vemurafenib or dabrafenib if the amount of BRAF V600E is similar to the amount of BRAF V600E in a similar sample from a patient with LCH or ECD and a BRAF V600E mutation who has not been treated.

15. The method of claim 14, wherein the first sample is obtained after diagnosis of LCH or ECD but before or at commencement of treatment with vemurafenib or dabrafenib and
continuing the treatment with vemurafenib or dabrafenib if the amount of BRAF V600E in the second sample is significantly lower than the amount of BRAF V600E in the first sample, or
discontinuing the treatment with vemurafenib or dabrafenib if the amount of BRAF V600E in the second sample is not significantly lower than the amount of BRAF V600E in the first sample.


SEQUENCE LISTING (2 sequences)

SEQ ID NO: 1 - DNA, 2946 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttcttttaaaaa

SEQ ID NO: 2 - protein, 766 aa, Homo sapiens [sequence omitted]